(19) United States
(12) Patent Application Publication
(10) Pub. No.: US 2023/0051357 A1
(43) Pub. Date: Feb. 16, 2023

(54) SHAVING SYSTEMS, METHODS, AND DEVICES

(71) Applicant: New Shave, LLC, Clermont, FL (US)

(72) Inventor: Thomas A. Kovatch, Clermont, FL (US)

(73) Assignee: New Shave, LLC, Clermont, FL (US)

(21) Appl. No.: 17/878,871

(22) Filed: Aug. 1, 2022

Related U.S. Application Data
(60) Provisional application No. 63/231,601, filed on Aug. 10, 2021.

(51) Int. Cl. A45D 27/29; A61K 8/92; A61Q 9/02

(52) U.S. Cl. CPC A45D 27/29; A61K 8/92; A61Q 9/02; A61K 2800/524; A61K 2800/526

(57) ABSTRACT

Shaving systems, methods, and devices, include a shaving device and a shaving device holder that prolongs a usable duration of the shaving device. The shaving device holder has a receptacle portion that holds a fluid (e.g. oil) which creates an oxygen free environment for a blade of the shaving device when the blade is submerged in the fluid. The system further includes components for preserving the fluid, such as a movable lid to guide water and debris away from an interior of the receptacle portion, and a wicking material to absorb water that ingresses into the interior the receptacle portion. A base plate attaches to an underside of the receptacle portion with one or more positioning protrusions shaped to fit the receptacle portion. Additionally or alternatively, a vertical backstop and/or shaving cover can enclose the shaving device and provide a surface for mounting the devices to an environmental surface.

### CROSS-REFERENCE TO RELATED APPLICATION

The present application claims priority under 35 U.S.C. § 119 to U.S. provisional patent application No. 63/231,601 filed Aug. 10, 2021, entitled “Shaving Apparatus,” which is specifically incorporated by reference in its entirety.

### BACKGROUND

Shaving devices come in many varieties, all with their own benefits and drawbacks. Common across most shaving devices is the difficulty in maintaining a sharp blade. The shaving razor typically dulls over multiple uses and needs regular replacing. As such, disposable razors have gained in popularity because the dulled blade is replaced with a new sharp blade at frequent intervals. However, disposable razors are wasteful and expensive to continuously replace and discard. It is with these observations in mind, among others, that the presently disclosed technology was conceived.

### BRIEF SUMMARY

In some instances, the systems, methods, and devices disclosed herein address the foregoing problems. For instance, a shaving system can include a shaving device with a razor head attached to a distal end of a handle. The shaving system can also include a shaving device holder that prolongs a usable duration of the shaving device. The shaving device holder can include a receptacle portion having an interior defined by one or more sidewalls; and a fluid contained in the interior of the receptacle portion such that the razor head is submerged in the fluid when the shaving device is held by the shaving device holder.

In some examples, the fluid prevents oxidization of the razor head thus prolonging the usable duration of the shaving device. For instance, the usable duration of the shaving device can be extended by using the fluid to form a corrosion free environment for the razor head in the receptacle portion. Additionally, the shaving device holder can further include a wicking material disposed at least partially in the interior of the receptacle portion to absorb water from the interior of the receptacle portion. The wicking material can by hydrophilic and the water absorbed by the wicking material can be evaporated into an environment above the oil. Furthermore, the wicking material can repel the fluid and/or the wicking material can be oleophobic.

In some instances, the shaving system further includes a movable lid positionable over a top opening of the shaving device holder. Furthermore, the one or more sidewalls can define a curved top edge of the receptacle portion; and the movable lid can have a curved surface corresponding to the curved top edge of the receptacle portion. The shaving system can further include a hinge coupling the movable lid to a front edge of the receptacle portion. Moreover, the movable lid can include a slot for receiving the handle when the shaving device is held by the shaving device holder.

In some examples, a shaving system comprises a shaving device including a razor head and a handle; and a shaving device holder that prolongs a usable duration of the shaving device. The shaving device holder can include a receptacle portion; a movable lid positionable over a top opening of the receptacle portion; and a fluid contained in the receptacle portion. The fluid can create an oxygen free environment for the razor head when the razor head is submerged in the fluid.

In some instances, the razor head is submerged in the fluid when the shaving device is held by the shaving device holder. The fluid can be a scented oil including a preservative ingredient. Furthermore, the shaving system can include a base plate adjacent to a bottom surface of the receptacle portion. The base plate can include one or more positioning protrusions extending from a top surface of the base plate which prevent lateral movement of the receptacle portion. Additionally or alternatively, the system can include a backstop extending from the base plate and having a distal edge that extends past a distal end of the handle when the shaving device is held by the shaving device holder. A shaving device cover can be removably couplable to the backstop to enclose the shaving device.

Furthermore, in some examples, the system includes one or more adhesive strips disposed on a back surface of the backstop. Additionally or alternatively, the one or more adhesive strips can be disposed on a bottom surface of the base plate. The system can also include a hydrophilic and/or oleophobic wicking material partially submerged in the fluid and partially outside the fluid.

In some instances, a shaving system includes a shaving device including a razor head and a handle; and a shaving device holder that prolongs a usable duration of the shaving device. The shaving device holder can include a receptacle portion; a movable lid positionable over a top opening of the receptacle portion; an fluid contained in the receptacle portion creating an oxygen free environment for the razor head when the razor head is submerged in the fluid; and a hydrophilic wicking material partially submerged in the fluid.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems, methods, and devices consistent with the disclosed subject matter and, together with the description, serves to explain advantages and principles consistent with the disclosed subject matter, in which:

FIG. 1 illustrates a perspective view of an example system including a shaving device and a shaving device holder with a movable lid;

FIG. 2. illustrates a perspective view of an example system including a shaving device and a shaving device holder with a backstop and a shaving device cover, which can form at least a portion of the system depicted in FIG. 1;

FIG. 3 illustrates a cross-sectional side view of an example system including a shaving device and a shaving device holder with a movable lid, which can form at least a portion of the system depicted in FIG. 1;

FIG. 4 illustrates a cross-sectional side view of an example system including a shaving device and a shaving device holder with a backstop and shaving device cover, which can form at least a portion of the system depicted in FIG. 1;

FIGS. 5A and 5B illustrate perspective views of a system including a baseplate and a receptacle portion of a shaving device holder, which can form at least a portion of the system depicted in FIG. 1; and

FIGS. 6A and 6B illustrate partial perspective views of a system including a movable lid of a shaving device holder transitionable between a closed position and an open position, which can form at least a portion of the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale, components may be omitted, and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

### II. General Architecture

Systems, methods, and devices disclosed herein include a shaving device and a shaving device holder. These devices, manufactured as discussed herein, can form a consumer product in which the shaving device holder extends the life of the razor by substantially eliminating oxidizing elements of air and water. Furthermore, the device(s) disclosed herein can coat the blade of the razor when the razor is taken out of the container, making the razor smooth and efficient for cutting hair.

The design of the shaving device holder submerges the blade in oil and prevents water from entering the container. The oil surrounds the blade providing an air, moisture, and oxygen/corrosive free environment which maintains the sharpness and extends the life or usable duration of the blade while also providing a smoother and more comfortable shave. Because the blade itself is stored in oil, no water or air can reach the blade element, thus preventing rust and corrosion associated with water and oxygen and giving the blade a much longer life. Additionally, a wicking mechanism can remove any water which gets into the oil. Furthermore, the oil can be a scented oil and/or can include preservatives. The scented and/or preserved oil can stay fresh and clean without going rancid or degrading from exposure to water, oxygen, and/or body oils or hair because the preservatives prevent oxidization, mold, degradation, or other chemical reactions that cause the oil to break down over time.

The different facets discussed herein can be combined in a blade storage unit which can be conveniently placed and/or secured on a counter top or shower wall among other locations (e.g., with an attachment mechanism such as an adhesive strip). As such, the razor blade can be placed away from other toiletries that are typically located on the counter top in a bathroom. Accordingly, the razor avoids being stored beside toothbrushes, other razors, bottles, picks, etc. which can nick the blade and further contribute to dulling of the blade.

The technology disclosed herein can overcome blade oxidization which causes pitting and dull shaves. Additionally, the systems provide a smooth shave every time by keeping the blade as close as possible to its original sharpness. When the shaver and blade are removed, the blade is fully lubricated and, therefore, provides a significantly smoother and closer shave while also lubricating the skin. In some instances, the shaving device disclosed herein can be used without shaving cream for an extended duration.

Additional advantages and benefits of the presently disclosed technology will become apparent from the detailed description below.

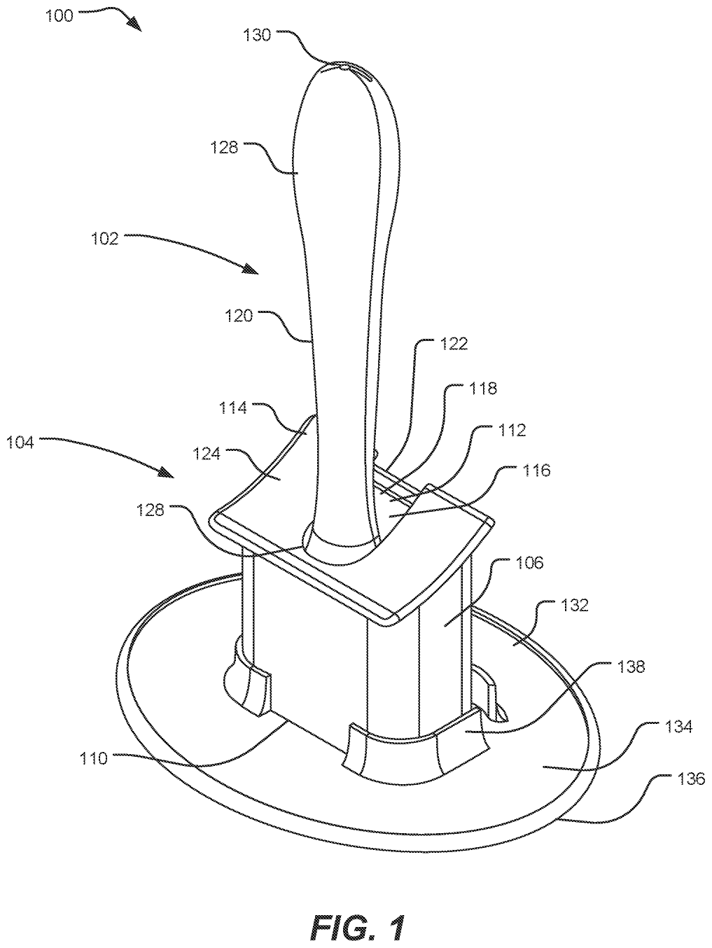

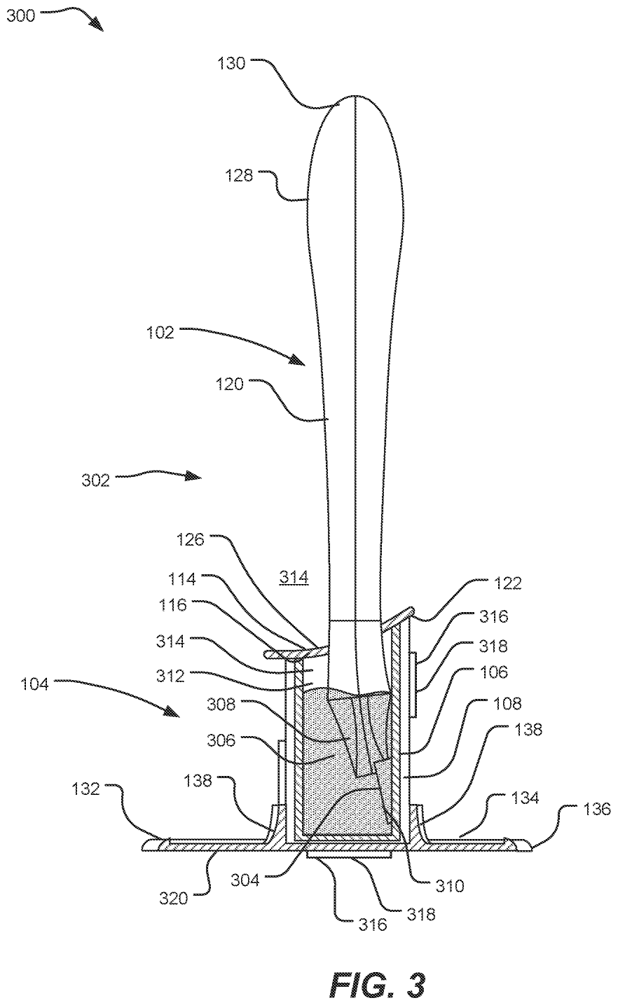

FIG. 1 illustrates an example system 100 including a shaving device 102 and a shaving device holder 104 that stores and/or holds the shaving device 102. The shaving device holder 104 can prolong a usable duration of the shaving device 102 by, for instance, forming an oxygen free environment for one or more portions of the shaving device 102. The shaving device holder 104 can include a receptacle portion 106 formed by one or more sidewalls 108 extending vertically from a bottom 110 of the receptacle portion 106. The interior portion of the receptacle portion 106 can hold a fluid, such as an oil, a scented and/or preservative enriched oil, vegetable oil, mineral oil, an organic oil, an inorganic fluid, combinations thereof, and so forth. The fluid can receive a razor head (e.g., razor head 304 depicted in FIG. 3) and, with the razor head submerged, create an oxygen free environment for the razor head while being stored between uses. Preventing oxidization of the metal blade can prevent pitting that dulls the blade. Furthermore, the oil can coat the blade(s) on the razor head when the shaving device 102 is taken out of the shaving device holder 104 so that the blade(s) have the additional benefit of smooth and efficient hair cutting.

In some examples, a corrosion free environment is formed and maintained by various components of the receptacle portion 106. For instance, a wicking material 112 can be disposed in the interior of the receptacle portion 106, at least partially submerged in the oil. The wicking material 112 can absorb water from the interior of the receptacle portion 106 and/or evaporate the water out of the receptacle portion 106. Additionally, the oil can include one or more essential oils, other scent ingredients, and/or preservatives to prevent oxidization, mold, degradation, or other chemical reactions that cause the oil to break down over time.

In some instances, the shaving device holder 104 includes a movable lid 114 or cover at least partially disposed over a top opening 116 of the receptacle portion 106, the top opening 116 being defined by one or more top edges of the one or more sidewalls 108. The movable lid 114 can include a slot 118 through which a handle 120 of the shaving device 102 can extend. The slot 118 can extend from a back edge 122 of the receptacle portion 106 through a top surface 124 of the movable lid 114, and can include a curved edge 126 to correspond to a curved surface of the handle 120. The handle 120 can extend through the slot 118 in the movable lid 114 and out of the shaving device holder 104 in a vertical direction. Moreover, the handle 120 can include an elongated center portion with a widened section 128 (e.g., having a width dimension greater than a width dimension of the slot 118) that tapers to a first distal end 130 of the handle 120. A second distal end (e.g., second distal end 308 in FIG. 3) of the handle 120 opposite the first distal end 130 can have the razor head attached.

In some instances, the shaving device 102 and shaving device holder 104 can include components for mounting or securing the shaving device 102 and/or shaving device holder 104 to different surfaces (e.g., in a bathroom, a bath, a shower, etc.). In some examples, the shaving device holder 104 includes a base plate 132 forming a planar surface 134 that mates with a bottom surface of the shaving device holder 104. The planar surface 134 of the base plate 132 can extend beyond the bottom surface of the shaving device holder 104 to an outer edge 136 of the base plate 132. One or more positioning protrusion(s) 138 can extend from the planar surface 134 with shapes corresponding to the outer perimeter of the shaving device holder 104 formed by the one or more sidewalls 108, such that the shaving device holder 104 is received by the positioning protrusion(s) 138 and lateral motion of the shaving device holder 104 is prevented by the positioning protrusion(s) 138. The base plate 132 can provide foundational stability for the shaving device holder 104 and the shaving device 102. Moreover, one or more adhesive strips or adhesives can be disposed on a bottom surface of the base plate 132, as discussed in greater detail below regarding FIG. 3.

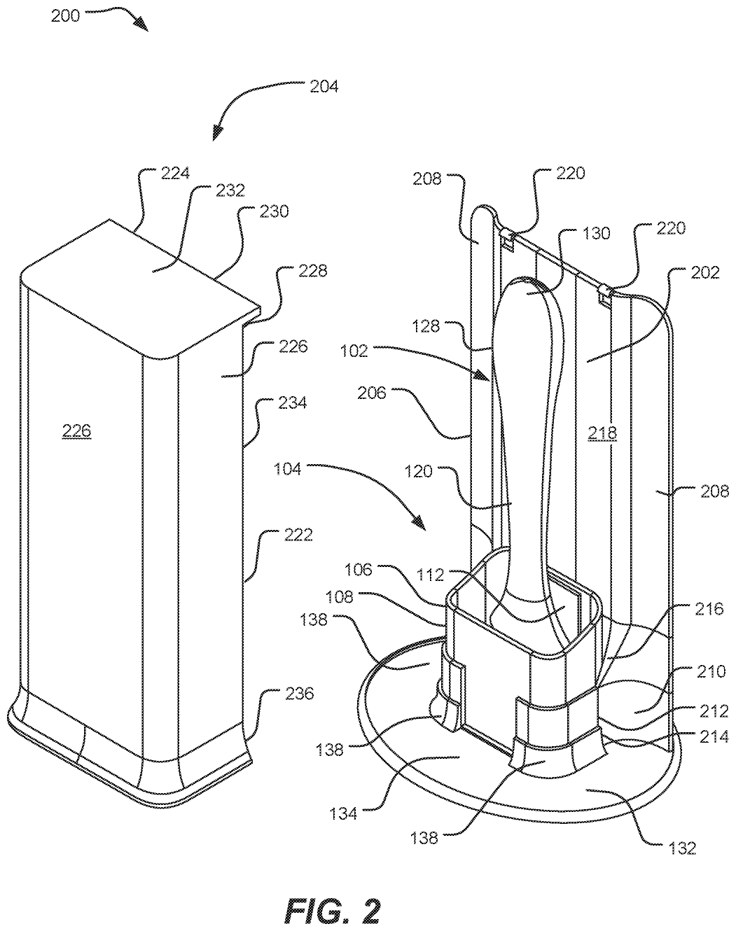

FIG. 2 illustrates an example system 200 including the shaving device 102 and the shaving device holder 104, which can form at least a portion of the system 100 depicted in FIG. 1. As depicted in FIG. 2, the shaving device holder 104 can include a backstop 202 and/or a shaver cover 204.

For instance, the backstop 202 can be a back wall or surface extending vertically from the base plate 132 and, in some examples, has a height 206 extending past the first distal end 130 of the handle 120. The backstop 202 can include one or more side wings 208 formed by a portion 210 of the backstop 202 extending from the receptacle portion 106. The one or more side wings 208 can extend the full height 206 or only a portion of the height 206 of the backstop 202, and can form a flat rear surface to mate with a vertical mounting surface (e.g., a shower wall, etc.). In some instances, a tape (e.g., a double sided, water resistant tape) can be placed on the rear surfaces of the one or more side wings 208 to mate to the vertical mounting surface and secure the shaving device 102 and the shaving device holder 104 in place. The backstop 202 can also include one or more connections or connection lines 212 coupling the backstop 202 to one or more rear edges 214 of the positioning protrusion(s) 138 extending from the base plate 132 (e.g., or to a rear surface of the receptacle portion 106). For instance, one or more tapered surfaces 216 can extend from a back plane 218 of the backstop 202, with smooth contouring between the back plane 218 and the receptacle portion 106, to mate along the one or more rear edges 214 and form the connection lines 212. In this way, the backstop 202 the base plate 132, and/or the protrusion(s) 138 can be integrated together as a single integrated unit. Additionally or alternatively, these components may be separable and/or interchangeable.

In some examples, the backstop 202 further includes one or more cover couplers 220, such as snap-fits, buttons, latches, hooks, and so forth for removably coupling the backstop 202 to the shaver cover 204. The shaver cover 204 can have a height 222 and/or a width 224 corresponding to the backstop 202 and one or more side surfaces 226. One or more baseplate couplers 228 can be disposed on the shaver cover 204, for instance, at a rear of the shaver cover 204 to mate with the one or more cover couplers 220 on the backstop 202, securing the shaver cover 204 to the backstop 202. The one or more baseplate couplers 228 can be positioned below a shelf 230 formed by a top surface 232 of the shaver cover 204 extending past back edge(s) 234 of the one or more side surfaces 226. Moreover, the shaver cover 204 can have an interior space formed by the one or more side surfaces 226 for receiving the shaving device 102 and the shaving device holder 104, and at least partially enclosing the shaving device 102 and the shaving device holder 104 when the shaver cover 204 attaches to the backstop 202 and/or the base plate 132. Additionally or alternatively, the backstop 202 can include a flared bottom 236 that corresponds with a curved or tapered shape of the positioning protrusion(s) 138. The backstop 202 can mate with the base plate 132 and enclose the positioning protrusion(s) 138. Furthermore, one or more handle mounting components can extend from the back plane 218 to hold or pinch the handle 120 (e.g., snap fits, hooks, foam blocks, and so forth).

As such, when the system 200 is in a stored state, the shaver cover 204 can be attached to the shaving device holder 104 via the backstop 202 and can at least partially and/or fully enclose the shaving device 102 and the receptacle portion 106, thus preventing water ingress to the corrosion free environment of the receptacle portion 106 (e.g., by covering the top opening 116 of the receptacle portion 106). Moreover, the shaver cover 204 can be easily removed via the two cover couplers 220 and corresponding one or more baseplate couplers 228 in order to remove the shaving device 102 from the shaving device holder 104.

FIG. 3 illustrates an example system 300 with a cross-sectional side view of the shaving device 102 and the shaving device holder 104 including the movable lid 114 over the top opening 116 of the receptacle portion 106. The system 300 depicted in FIG. 3 can form at least a portion of the system 100 depicted in FIG. 1.

In some examples, the system 300 shows the shaving device 102 in a stored state 302 with a razor head 304 stored and the movable lid 114 closed over the top opening 116. Also, in the stored state, the receptacle portion 106 can maintain the razor head 304 submerged in an oil 306 (e.g., vegetable oil, coconut oil, and so forth). The razor head 304 coupled to a second distal end 308 of the handle 120 can be held in place by a form-fitted interior surface of the receptacle portion 106 (e.g., a snap fit, a compression fit, a receiving slot, an indent, a bump or protrusion, and so forth). As such, some or all of the metallic blade portions 310 of the razor head 304 can be submerged in the oil 306, below a surface interface 312 of the oil 306 with outside air 314. This can prevent rust from growing on the metallic blade portions 310 while also preventing corrosion of the sharp blade edge. Moreover, when the shaving device 102 is being used, the movable lid 114 can prevent water, hair, soap, body fluid, skin particles, and so forth from falling into the oil 306 to keep the oil fresh.

In some instances, the shaving device holder 104 can include one or more surface mounts 316 for removably and/or securely fixing the shaving device holder 104 in place on one or more environmental surfaces. For instance, an adhesive strip 318 can extend from a bottom surface 320 of the base plate 132 and/or of the receptacle portion 106. The one or more surface mounts 316 can include a double-sided adhesive strip 318, tape a water resistant material, a snap-fit, a hook, one or more suction cups, combinations thereof. The one or more surface mounts 316 may be positioned on the bottom surface 320 of the base plate 132 and/or of the receptacle portion 106 for attaching the shaving device holder 104 and the shaving device 102 to a horizontal surface (e.g., a shower surface, a bath surface, a counter surface, and so forth).

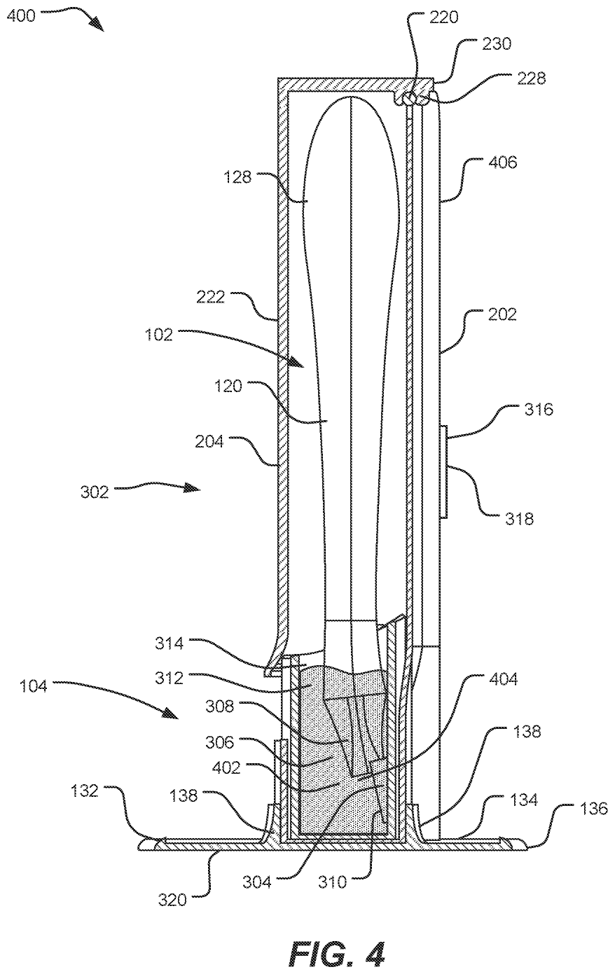

FIG. 4 illustrates an example system 400 with a cross-sectional side view of the shaving device 102 and the shaving device holder 104 including the backstop 202 and the shaver cover 204. The system 400 can form at least a part of the system 100 depicted in FIG. 1.

In some instances, the system 400 shows the shaving device 102 in the stored state 302 with the shaver cover 204 and the backstop 202 enclosing the shaving device 102 and the receptacle portion 106 of the shaving device holder 104. The backstop 202 can removably couple to the shaver cover 204 holding the shaver cover 204 in place over the shaving device 102 and the shaving device holder 104. This arrangement can contribute to forming a corrosion free environment 402 for the razor head 304 by preventing water, hair, skin cells, other contaminants and the like from falling into the receptacle portion 106 when the shaving device holder 104 and the shaving device 102 are in the stored state. The oil 306 can also form the corrosion free environment 402 and/or oxidization free environment 404 by preventing contact between the blade(s) of the razor head 304 and oxygen when the razor head 304 is submerged in the oil 306. Furthermore, placing the razor head 304 in the oil 306 can cause hair, skin, and body fluids on the razor head 304 to be cleaned from the razor head 304, further preventing corrosion.

In some instances, the shaving device holder 104 can include the one or more surface mounts 316 for removably and/or securely fixing the shaving device holder 104 in place on one or more environmental surfaces. For instance, the adhesive strip 318 can extend from a back surface 406 of the backstop 202 (e.g., at the one or more side wings 208). The one or more surface mounts 316 can include a double-sided adhesive strip, tape a water resistant material, a snap-fit, a hook, one or more suction cups, combinations thereof, and so forth. The surface mount(s) 316 may be positioned on the back surface 406 of the backstop 202 for attaching the shaving device holder 104 and the shaving device 102 to a vertical surface (e.g., a shower surface, a bath surface, a wall surface, and so forth). In some examples, when the shaving device holder 104 includes the backstop 202, the system 400 can omit the base plate 132. For instance, the base plate 132 the backstop 202, the shaver cover 204 and/or the movable lid 114 can be removably coupled to the receptacle portion 106 (e.g., via friction fits) so that these components are interchangeable and the shaving device holder 104 can be reconfigurable into a horizontal mounting configuration (e.g., with the base plate 132) and/or a vertical mounting configuration (e.g., with the backstop 202 and/or the shaver cover 204).

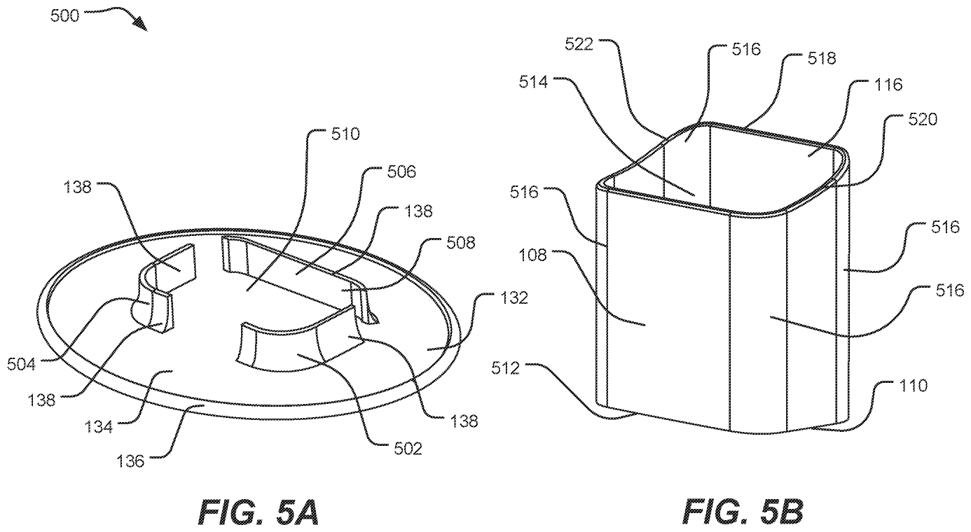

FIGS. 5A and 5B illustrate an example system 500 including the base plate 132 of the and the receptacle portion 106 of the shaving device holder 104. The system 500 can form at least a portion of the system 100 depicted in FIG. 1.

In some examples, the base plate 132 can have one or more positioning protrusion(s) 138 extending from the planar surface 134. The positioning protrusion(s) 138 can form an outline or a partial outline of a top-view perimeter shape of the receptacle portion 106. In other words, the positioning protrusion(s) 138 can form a shape corresponding to a shape of the receptacle portion 106. The positioning protrusion(s) 138 can include a first corner protrusion 502, a second corner protrusion 504, and any number of corner protrusions to match a shape of one or more corners of the receptacle portion 106 (e.g., rounded corners or squared corners). Additionally or alternatively, the positioning protrusion(s) 138 can include a side protrusion 506, such as a rear side protrusion 508, which can be linear and correspond to the one or more sidewalls 108 of the receptacle portion 106. As such, the positioning protrusion(s) 138 can form at least partially a triangle, rectangle, square, circle, or any other shape corresponding to the receptacle portion 106. An inner bottom surface 510 at least partially surrounded by the positioning protrusion(s) 138 can mate with a bottom surface 512 of the receptacle portion 106. Moreover, the inner bottom surface 510 can include one or more of an adhesive, tape, friction-fit, indents, protrusions, etc., for at least partially securing the receptacle portion 106 to the base plate 132.

Furthermore, the base plate 132 can have the outer edge 136 extending past the one or more sidewalls 108. As such, the base plate 132 can expand the footprint of the shaving device holder 104, thus increasing stability for the shaving device holder 104.

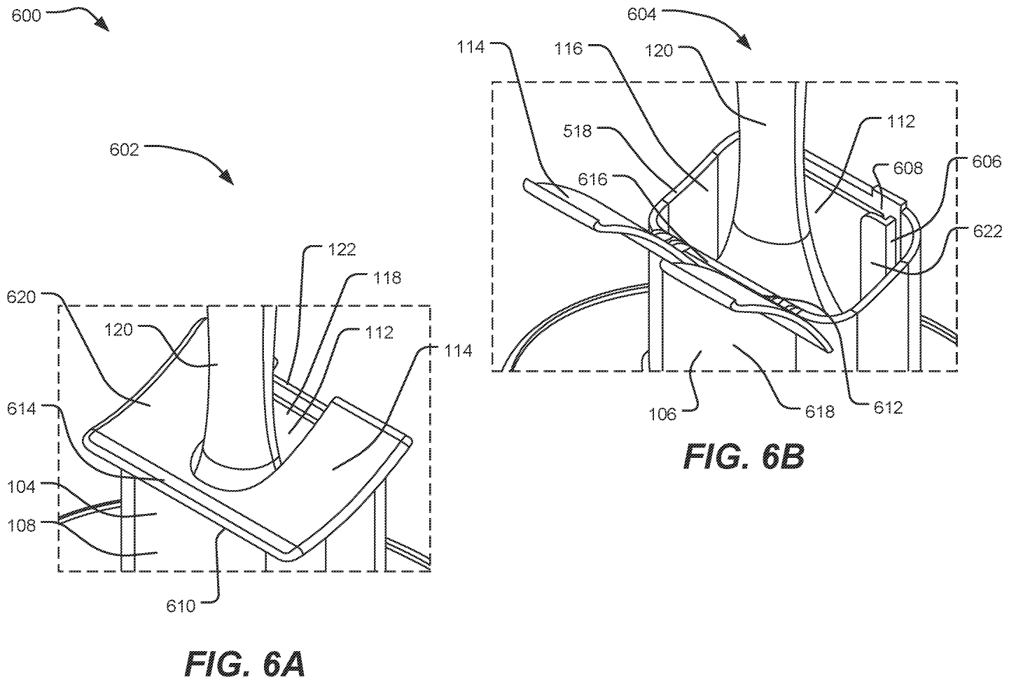

Turning to FIG. 5B, the receptacle portion 106 can be formed by the one or more sidewalls 108 which define an interior space 514. The receptacle portion 106 can have a generally rectangular base/top view profile with rounded corners 516. In some scenarios, the one or more sidewalls 108 can form other profiles, such as a circular profile, a square profile, and the like. A top edge 518 can be formed by the vertical termination of the one or more sidewalls 108. The top edge 518 can include a first side curve 520 and a second side curve 522, which can correspond with and/or mate with a curved surface (e.g., curved surface 620 discussed below regarding FIGS. 6A and 6B) of the movable lid 114. The receptacle portion 106 can have various indents and/or snap fits for mating with the other components of the system 100, such as the base plate 132, the positioning protrusion(s) 138, the backstop 202, the movable lid 114, and the like. Moreover, the receptacle portion 106 can include one or more indents, snap-fits, or other components for securing the razor head 304 and/or the second distal end 308 of the shaving device 102.

FIGS. 6A and 6B illustrate an example system 600 including the movable lid 114 transitionable between a closed position 602 and an open position 604. FIG. 6B further illustrates the wicking material 112 held in place by a wicking material mount 606 on an interior surface 608 of the receptacle portion 106. The system 600 can form at least a portion of the system depicted in FIG. 1.

In some examples, the movable lid 114 is hingedly coupled to the top edge 518 (e.g., top front edge 610) of the receptacle portion 106. One or more hinges 612 (e.g., one, two, three, or four) can attach a front edge 614 of the movable lid 114 to a front top edge 616 of the receptacle portion 106 (e.g., formed by a terminating end of a front sidewall 618). As such, the movable lid 114 can swing up and away from the top opening 116 of the receptacle portion 106. The one or more hinges 612 can have a predetermined swing range to prevent the movable lid 114 from swinging beyond a certain angle. Moreover, the one or more hinges 612 can be spring-loaded to provide a force maintaining/assisting in the closed position 602.

In some instances, when the movable lid 114 is in the closed position 602 the movable lid 114 can prevent water from entering the receptacle portion 106, maintaining a cleanliness of the oil 306. This corrosion free environment 402 can be maintained with the shaving device 102 in the stored state 302 (e.g., with the razor head 304 submerged in the oil 306) as well as when the shaving device 102 is being used and/or the shaving device holder 104 is not being used to hold the shaving device 102. Furthermore, the movable lid 114 can include a curved surface 620 (e.g., concave facing up) extending from the back edge 122 of the movable lid 114 to the front edge 614. The curved surface 620 can match or correspond to one or more curved side top edges (e.g., the first side curve 520 and the second side curve 522) of the receptacle portion 106 so that the movable lid 114 forms a seal with the top edges 518 of the receptacle portion 106. In some scenarios, the curved surface 620 guides water from the environment (e.g., the shower) off of the shaving device holder 104 down to the front and away from the interior space 514. The movable lid 114 can include one or more overhangs extending past the one or more sidewalls 108 to guide water and other debris away from the interior space 514.

Furthermore, in some examples, the shaving device holder 104 includes the wicking material mount 606 in an interior rear surface of the receptacle portion 106. The wicking material mount 606 can include a holding member 622 forming a slot (e.g., a vertical slot) to receive the open position 604, as well as one or more protrusions, horizontal shelves, or stops extending from the interior rear surface. As such, the wicking material mount 606 can maintain the wicking material 112 in a mounted position on the interior rear surface without sagging (e.g., even when exposed to the oil 306). Accordingly, the wicking material 112 can have a lower portion submerged in the oil 306 and/or an upper portion above the oil 306 such that water can be absorbed from the oil 306 by the lower portion and evaporated out of the wicking material 112 at the upper portion.

It is to be understood that the specific order or hierarchy of steps in the methods depicted throughout this disclosure are instances of example approaches and can be rearranged while remaining within the disclosed subject matter. For instance, any of the operations or steps for manufacturing and/or using the shaving device 102 and shaving device holder 104 depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A shaving system comprising:
a shaving device including a razor head attached to a distal end of a handle; and
a shaving device holder that prolongs a usable duration of the shaving device, the shaving device holder including:
a receptacle portion having an interior defined by one or more sidewalls; and
an fluid contained in the interior of the receptacle portion such that the razor head is submerged in the fluid when the shaving device is held by the shaving device holder.

2. The shaving system of claim 1,
wherein,
the fluid prevents oxidization of the razor head thus prolonging the usable duration of the shaving device.

3. The shaving system of claim 1,
wherein,
the usable duration of the shaving device is extended by using the fluid to form a corrosion free environment for the razor head in the receptacle portion.

4. The shaving system of claim 1,
wherein,
the shaving device holder further includes a wicking material disposed at least partially in the interior of the receptacle portion to absorb water from the interior of the receptacle portion.

5. The shaving system of claim 4,
wherein,
the water absorbed by the wicking material is evaporated into an environment above the fluid.

6. The shaving system of claim 4,
wherein,
the wicking material repels the fluid.

7. The shaving system of claim 1,
further including,
a movable lid positionable over a top opening of the shaving device holder.

8. The shaving system of claim 7,
wherein,
the one or more sidewalls define a curved top edge of the receptacle portion; and
the movable lid has a curved surface corresponding to the curved top edge of the receptacle portion.

9. The shaving system of claim 7,
further including,
a hinge coupling the movable lid to a front edge of the receptacle portion.

10. The shaving system of claim 7,
wherein,
the movable lid includes a slot for receiving the handle when the shaving device is held by the shaving device holder.

11. A shaving system comprising:
a shaving device including a razor head and a handle; and
a shaving device holder that prolongs a usable duration of the shaving device, the shaving device holder including:
a receptacle portion;
a movable lid positionable over a top opening of the receptacle portion; and
a fluid contained in the receptacle portion creating an oxygen free environment for the razor head when the razor head is submerged in the fluid.

12. The shaving system of claim 11,
wherein,
the razor head is submerged in the fluid when the shaving device is held by the shaving device holder.

13. The shaving system, of claim 11,
wherein,
the fluid is a scented oil including a preservative ingredient.

14. The shaving system of claim 11,
further including,
a base plate adjacent to a bottom surface of the receptacle portion, the base plate including one or more positioning protrusions extending from a top surface of the base plate which prevent lateral movement of the receptacle portion.

15. The shaving system of claim 14,
further including,
a backstop extending from the base plate and having a distal edge that extends past a distal end of the handle when the shaving device is held by the shaving device holder; and
a shaving device cover removably couplable to the backstop to enclose the shaving device.

16. The shaving system of claim 15,
further including,
one or more adhesive strips disposed on a back surface of the backstop.

17. The shaving system of claim 14,
further including,
one or more adhesive strips disposed on a bottom surface of the base plate.

18. The shaving system of claim 11,
further including,
a hydrophilic wicking material partially submerged in the fluid and partially outside the fluid.

19. The shaving system of claim 11,
further including,
an oleophobic wicking material partially submerged in the fluid and partially outside the fluid.

20. A shaving system comprising:
a shaving device including a razor head and a handle; and
a shaving device holder that prolongs a usable duration of the shaving device, the shaving device holder including:
a receptacle portion;
a movable lid positionable over a top opening of the receptacle portion;
an fluid contained in the receptacle portion creating an oxygen free environment for the razor head when the razor head is submerged in the fluid; and
a hydrophilic wicking material partially submerged in the fluid.
